(19) United States
(12) Patent Application Publication
(10) Pub. No.: US 2019/0336693 A1
(43) Pub. Date: Nov. 7, 2019

(54) DEVICE FOR INJECTING A CONTROLLED DOSE OF SOLUTION, RELATIVE PRODUCTION METHOD AND KIT

(71) Applicant: OROFINO PHARMACEUTICALS GROUP SRL, ROMA (IT)

(72) Inventor: Ernesto OROFINO, ROMA (IT)

(21) Appl. No.: 16/466,784

(22) PCT Filed: Nov. 29, 2017
(86) PCT No.: PCT/IB2017/057480

§ 371 Date: Jun. 5, 2019

(30) Foreign Application Priority Data
Dec. 5, 2016 (IT) 102016000123005

(51) Int. Cl. A61M 5/315; A61M 5/00

(52) U.S. Cl. CPC A61M 5/31501; A61M 5/002; A61M 2207/00; A61M 2005/3154; A61M 2005/31508; A61M 5/31591

(57) ABSTRACT

An injection device (100) for the injection of a controlled dose of an injectable substance, including: one containment chamber (33) of the injectable substance, defined inside a tubular body (3); a plunger (5) slidingly engaged inside the tubular body (3), provided with a central stem (53) placed between a cap (51) and a base (52); a spacer applied to the plunger (5) and adapted to allow a forward movement of the plunger (5) in the containment chamber (33) up to reach an abutment of the spacer between the base (52) of the plunger (5) and the second end (32) of the containment body (3) to prevent the further forward movement of the plunger (5) in the containment chamber (33). The injection device (100) is characterized in that the spacer is a flexible dosing label (9), that can be deformed in one longitudinal direction (X) and rigid in one transverse direction (Y).

The present invention relates to a device for injecting a solution, in particular a syringe provided with an element for controlling the quantity of solution to be delivered.

In the pharmaceutical and medical devices field a very relevant aspect to be taken into account is the correct dosage of the solution to be delivered. Traditional syringes are provided with a containment chamber wherein the solution to be injected is contained. Typically, the maximum injectable dose of solution is determined by the volume of the containment chamber when the plunger is completely withdrawn. In case a lower dose of solution needs to be injected, the user withdraws only partially the plunger until the desired dose is contained inside the containment chamber, or he withdraws the plunger completely and then moves it forward thus expelling the exceeding dose. The measurement of the volume of the solution inside the containment chamber is provided solely by a graduated scale traditionally present on the body of the syringe, usually made of transparent material.

In the field some tools are known, called spacers, for controlling more carefully the dose of administrable solution through a syringe. Spacers are elements of pre-established length, arranged around the plunger of the syringe. In use, the spacer abuts between the plunger end and the end of the containment body of the syringe, preventing the plunger from moving further forward. Consequently, only a known portion of the total volume of solution contained in the containment chamber is administered, and the quantity of drug to be administered depends on the length of the spacer.

An example of known spacer is disclosed in document EP1559443. The device in EP1559443 is a rigid body composed of two portions joined by a hinge. The device can be closed around the plunger of the syringe through a snap-lock mechanism. The device is rigid, made of plastic, for example by injection moulding.

Known spacers have some drawbacks. since they are complex devices, including hinges and snap-lock elements, they require dedicated moulds resulting in high production costs. Furthermore, applying them on the plunger of the syringe, in case they are assembled directly in the factory, requires the use of specific expensive and bulky machinery.

The need perceived in the field of injection devices is to have available a syringe provided with an element for controlling the quantity of solution to be delivered which is simple, cheap and easy to be assembled on the plunger.

The objective of this invention is overcoming the drawbacks of the prior art taking into account the needs of the field.

Such objective is achieved by an injection device provided with an element for controlling the quantity of solution to be delivered, in form of flexible label.

Furthermore, the solution according to this invention is particularly advantageous as the adjustment element in form of flexible label can be easily applied on the plunger of the injection device through a simple labeller.

Such objective is achieved by an injection device according to claim 1, a relative production method according to claim 13, and an injection kit according to claim 14. Dependent claims disclose preferred or advantageous embodiments of the device.

The features and the advantages of the injection device according to the present invention are apparent by the hereinafter reported disclosure, for exemplary and not limiting purposes, according to the enclosed figures, wherein:

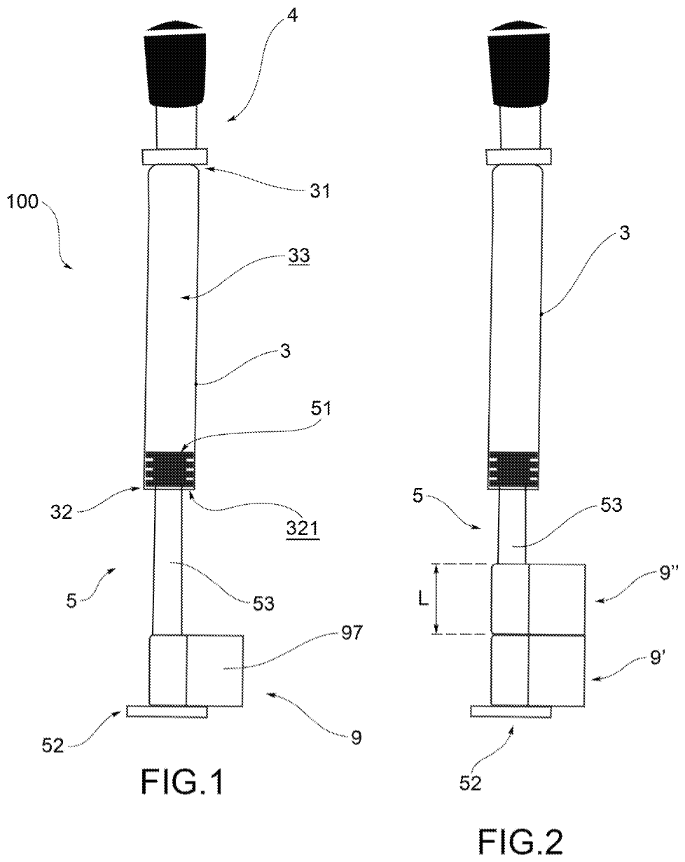

FIG. 1 shows a front view of an injection device according to the present invention, provided with an element for controlling the quantity of solution to be delivered in form of label (hereinafter dosing label);

FIG. 2 shows the injection device of FIG. 1 provided with more than one dosing labels;

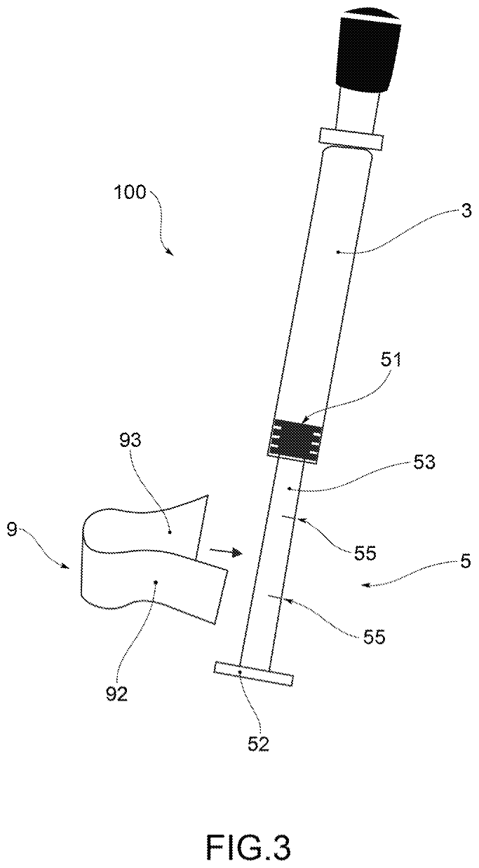

FIG. 3 shows a front view of the back face of the dosing label of FIG. 1, in a configuration detached from the injection device;

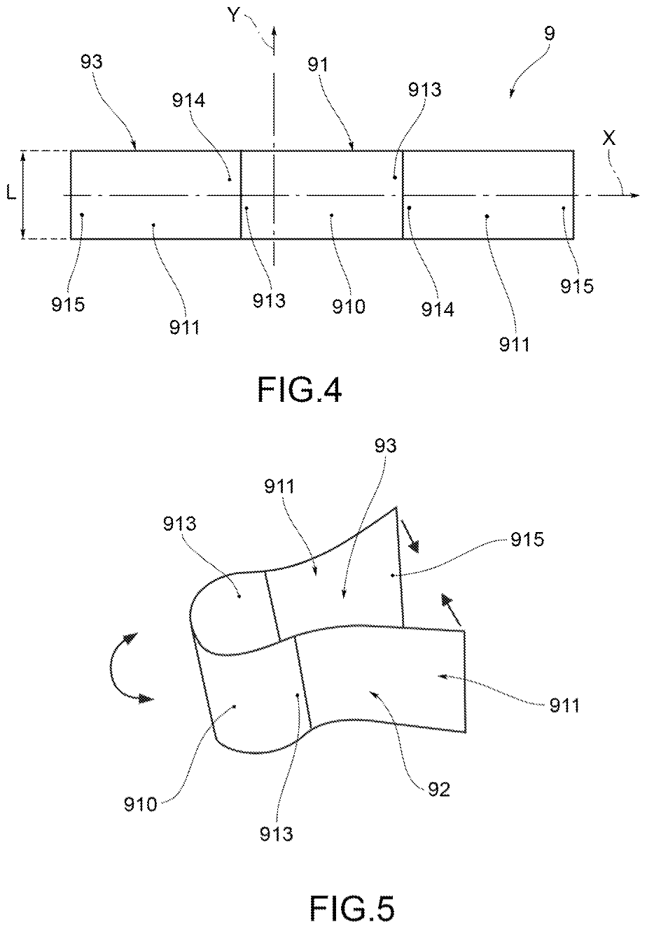

FIG. 4 shows a planar view of the dosing label of FIG. 1, in a planar configuration;

FIGS. 5 to 7 show some application steps of the dosing label on the plunger of the injection device, in particular the step of positioning the dosing label on the plunger, the step of closing the dosing label around the plunger, the step of securing it in position around the plunger;

FIG. 8 shows the step of using the injection device provided with a dosing label according to the present invention.

Referring to the enclosed figures, and in particular to FIG. 1, an injection device is indicated with reference number 100.

In the embodiment illustrated in the above described Figures, the injection device is a syringe.

The injection device 100 includes a tubular containment body 3, extending between one first end 31 that defines a front opening, and one second end 32 that defines a back opening 321.

The tubular body 3 is the containment body of the syringe, adapted to contain one or ore injectable substances.

Preferably, the tubular body 3 is made of glass or of a transparent plastic material or substantially transparent. Preferably, the tubular body 3 is made in a single piece.

The front opening 31 is adapted to allow the outflow of an injectable solution.

Preferably, the front opening 31 of the tubular body 3 is closed by a removable cap 4, adapted to seal the tubular body 3 on the lower part until when the injection device 100 is used. In particular FIG. 1 shows the sterile and ready-for-use injection device 100, wherein the front opening of the tubular body 3 is closed by the removable cap 4.

Once the cap 4 has been removed, the front opening of the tubular body 3 is engageable with a needle adapted to allow injecting the injectable solution. In particular, when the injection device 100 is ready for the injection, the needle is in fluid communication with the front opening of the tubular body 3.

The injection device 100 includes at least one containment chamber 33, defined inside the tubular body 3, for containing one injectable substance.

The injection device 100 includes one plunger 5 slidingly engaged inside the tubular body 3.

The plunger 5 includes a central stem 53, provided with one cap 51 on one side, and a base 52 on the opposite side. As clearly shown in FIGS. 1-3 and 6-8, the base 52 preferably projects laterally, for example radially, from the stem 53. The base 52 is for example a plate, preferably of circular or elliptic shape.

The cap 51, arranged inside the containment chamber 33, is adapted to slide inside the tubular body 3 of the syringe due to a pushing or traction force exerted by the plunger 5.

Cap 51 is for example made of rubbery and/or plastic material and it is such as to sealingly engage with the inner walls of the tubular body 3 of the injection device 100, and it is such as to be able to slide inside the injection device 100 to allow the outflow of the injectable solution.

The base 52 of the plunger 5 provides a supporting or grasping point for the user, for moving forward or backward the plunger 5 with respect to the tubular body 3 of the injection device 100.

The maximum injectable dose of solution through the injection device 100 is determined by the total volume of the containment chamber 33 when the plunger 5 is completely withdrawn. In case a lower dose of solution needs to be injected, the user withdraws only partially the plunger 5 until the desired dose is contained inside the containment chamber 8, or he withdraws completely the plunger 5 and then moves it forward thus expelling the exceeding dose.

In order to control more carefully the administrable dose of solution, the injection device 100 includes one spacer 9.

The spacer 9 is an element of pre-established length L, arranged around the plunger 5, in particular placed around the central stem 53. In use, the spacer 9 abuts between the base 52 of the plunger 5 and the second end 32 of the containment body 3 of the syringe, preventing the plunger 5 from moving further forward inside the containment chamber 33. Consequently, only a known portion of the total volume of solution contained in the containment chamber 33 is administered, and the quantity of drug to be administered depends on the length L of the spacer 9.

The injection device 100 includes a spacer in form of flexible label, hereinafter dosing label 9. The dosing label 9 is shown in detail in FIGS. 3-5.

The dosing label 9 is composed of one body 91, preferably of substantially rectangular and elongated shape. The width of the body 91 defines the predetermined length L of the spacer.

The body 91 includes a front face 92 and a back side 93. The body 91 includes a central portion 910, arranged between two side portions or ends 911. The central portion 910 is meant, while the injection device is in use, to abut between the base 52 of the plunger 5 and the second end 32 of the containment body 3 of the syringe.

The dosing label 9 is a strip of flexible material, preferably made of plastic material.

Advantageously, the dosing label 9 is a simple element to be manufactured and can be easily applied on the plunger 5 of the injection device 100 by means of a simple labeller.

In one embodiment, the body 91 is a strip of flexible material, that can be deformed in the longitudinal direction X and rigid in the transverse direction. In other words, it is capable of flexing and closing as in FIG. 5, and capable of enduring the shear strengths as in FIG. 8. In fact, in use, the dosing label 9 abuts between the base 52 of the plunger 5 and the second end of the containment chamber 3 of the syringe, preventing the plunger 5 from moving further forward inside the containment chamber 33.

In one further embodiment, the body 91 is a strip of flexible material, that can be deformed in the longitudinal direction X and rigid in the transverse direction only at the central portion 910.

Preferably, the central portion 910 is more rigid than the ends 911, though it can always be deformed in the longitudinal direction X to allow the folding of the dosing label 9 as in FIG. 4.

In one example, the central portion 910 is implemented with a thicker body portion 91, having indeed greater thickness than the ends 911.

In one further example, the central portion 910 is implemented applying a plastic little bar in the middle of the body 91.

In one embodiment, the dosing label 9 can be plastically deformed in longitudinal direction, in other words, it maintains a C-form when it is folded as in FIG. 4.

In one further embodiment, the dosing label 9 can be plastically deformed only at the central portion 910, i.e. it maintains a C-form when it is folded as in FIG. 4.

The dosing label 9 can be closed as a ring when it is folded. In particular, the dosing label 9 can be closed at the ends 911.

The dosing label 9 is at least partially self-adhesive at the back side 93. In other words, at the back side 93 of the dosing label 9 a strip of adhesive material is applied, for example a glue.

In one embodiment, the back side 93 of the dosing label 9 is self-adhesive only at the ends 911 (i.e., the back side 93 of the dosing label 9 is free from adhesive material at the central portion 910). Once the dosing label 9 is folded in C-form, the ends 911 join between them at the adhesive back side, locking the dosing label 9 on the plunger 5.

In one further embodiment, the whole back side 93 of the dosing label 9 is adhesive. Thereby the dosing label 9 sticks on the plunger 5 and in particular on its central stem 53, at least at the central portion 910.

Preferably, the length (measured along the axis) of the central portion 910 of the dosing label 9 is smaller than the circumference of the plunger 5 of the injection device 100.

Such solution facilitates the folding of the dosing label 9 on the plunger 5 when there is a central portion 910 more rigid than the ends 911: in fact, in such way the rigid distal edges 913 of the central portion 910 do not interfere one another during the folding step of the label 9.

Such solution allows to glue the dosing label 9 at least partially to the plunger 5 also in case the central portion 910 is not provided with adhesive: in fact in that way the adhesive proximal edges 914 of the ends 911 are joined to the plunger 5 locking the label 9 in position.

Further preferably, the plunger 5 is graduated, i.e. it is provided with notches 55 at the stem 53 adapted to indicate to the user the correct positioning wherein the dosing label 9 must be applied.

Further preferably, the stem 53 of the plunger 5 has a circular section to facilitate the gluing in position of the dosing label 9.

FIG. 1 shows one injection device 100 provided with a dosing label 9 fixed in position on the stem 53 of the plunger 5. The dosing label 9 is fastened in abutment against the base 52 of the plunger 5.

FIG. 2 shows an injection device 100 provided with a plurality of dosing labels 9 (in particular two dosing labels 9) fastened in series along the stem 53 of the plunger 5. One first dosing label 9′ is fastened in abutment against the base 52 of the plunger 5, and one second dosing label 9″ is fastened in abutment against the first dosing label 9′. In such embodiment, the dosing labels 9 can be removed: once the injection of one first dose of solution has been performed, the second dosing label 9″ is removed in order to allow injecting one further dose of solution; it is possible to perform the injection of the residual dose of solution removing also the first dosing label 9′.

FIGS. 5 to 7 show some application steps of the dosing label 9 on the plunger 5 of the injection device 100. FIG. 5 shows the folding step of the dosing label 9 along direction X, to perform a C-form (or a U-form), for the following application on the injection device 100.

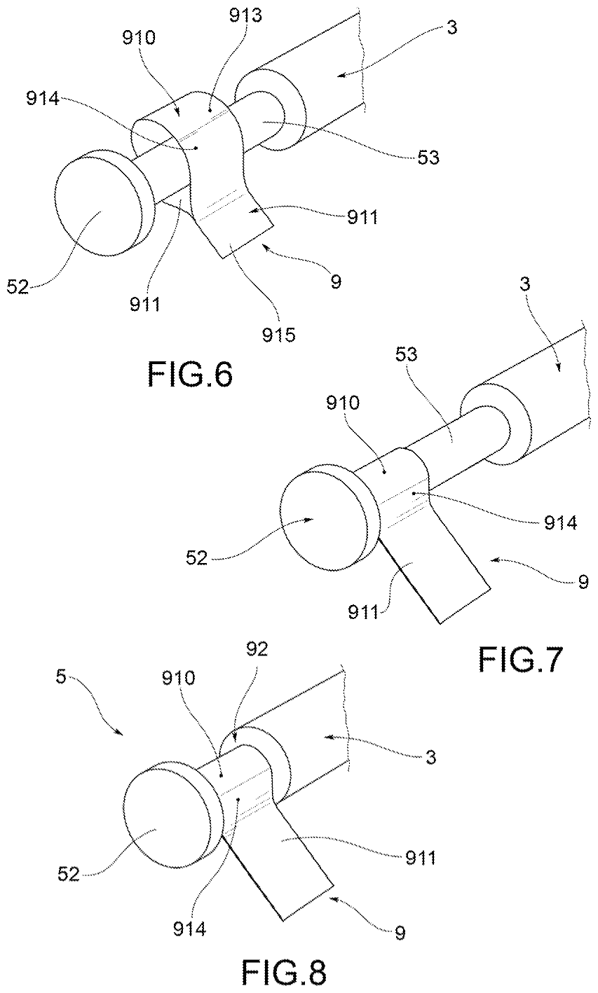

FIG. 6 shows the closing step of the dosing label 9 around the stem 53: distal edges 915 of ends 911 are joined between them (at the adhesive back side 93) so as to close the label 9 without preventing it from sliding along the plunger 5.

FIG. 7 shows a step of locking in position of the flexible label 9 around the plunger 53: the dosing label 9 is slided along the stem 53 until its abutment against base 52 of the plunger 5; adhesive proximal edges 914 of the ends 911 are joined to the stem 53 so as to glue the label 9 firmly in position on the plunger 5.

Advantageously, once fastened in position on the plunger 5, the dosing label 9 has a grasping portion 97 at the ends 911 overlapped and glued between them.

FIGS. 7 and 8 show the steps of use of the injection device 100 provided with a dosing label 9.

FIG. 7 shows an injection device 100 ready to be used: the forward movement of the plunger 5 determines the beginning of the delivery of the solution contained in the containment chamber 3.

The plunger 5 moves forward until when the dosing label 9 reaches and abutment between the base 52 of plunger 5 and the second end 32 of the containment body 3 of the injection device 100.

FIG. 8 shows the injection device 100 at the end of the injection: the further forward movement of the plunger 5 in the containment chamber 33, and the further delivery of the solution is in fact prevented by the presence of the dosing label 9.

Therefore, it is clear that the spacer applied to the plunger 5 is adapted to allow a forward movement of the plunger 5 in the containment chamber 33 up to reach of an abutment of the spacer between the plunger 5 and the second end 32 of the containment body 3, to prevent a further forward movement of the plunger 5 in the containment chamber 33.

An object of the present invention is an injection device 100 provided with at least one dosing label 9 applied on the plunger 5. In such a solution, the dosing label 9 is applied on the plunger 5 during the manufacturing step of the injection device 100 by means of a labeller.

An object of the present invention is also an injection kit including an injection device 100 and at least one dosing label 9 applicable on the plunger 5. In such solution, the dosing label 9 is applied on the plunger 5 directly by the user when the injection device 100 is in use.

An object of the present invention is also one production method for an injection device 100, including the step of applying the dosing label 9 on the plunger 5 by means of a labeller.

Innovatively, an injection device provided with dosing label according to the present invention is easy and cheap to manufacture.

Advantageously in fact, as the dosing label is a flexible label, it is easy to manufacture with no need for proper moulds.

Further advantageously, as the dosing label is a flexible label, it can be easily applied on the plunger of the injection device by a simple labeller.

Without prejudice to the principle of the invention, the embodiments and the implementation details shall be widely varied with respect to what has been disclosed and illustrated for non-limiting exemplary purposes, without departing from the scope of protection as defined in the enclosed claims.

1. An injection device (100) for injecting a controlled dose of an injectable solution, including:
a tubular containment body (3), extending between one first end (31) that defines a front opening, and one second end (32);
at least one containment chamber (33), defined inside the tubular body (3), for containing the injectable substance;
one plunger (5) slidingly engaged inside the tubular body (3), having a central stem (53) placed between a cap (51) and a base (52), said cap (51) being slidingly arranged inside the containment chamber (33) to allow the outflow of the injectable solution;
a spacer applied to the plunger (5) and adapted to allow a forward movement of the plunger (5) in the containment chamber (33) up to reach an abutment of the spacer between the base (52) of the plunger (5) and the second end (32) of the containment chamber (3) to prevent the further forward movement of the plunger (5) inside the containment chamber (33);
characterized in that the spacer is a flexible dosing label (9), that can be deformed in one longitudinal direction (X) and rigid in one transverse direction (Y).

2. An injection device (100) according to claim 1, wherein the dosing label (9) is a strip of flexible plastic material.

3. An injection device (100) according to claim 1, or wherein the dosing label (9) includes a central portion (910) placed between two ends (911), and wherein the central portion (910) is more rigid than the ends (911), though it can always be deformed in longitudinal direction (X).

4. An injection device (100) according to claim 3, wherein the central portion (910) has a thickness greater than the thickness of the ends (911).

5. An injection device (100) according to claim 3, wherein the dosing label (9) is composed of a body (91) and the central portion (910) is manufactured by applying a plastic bar at the middle of the body (91).

6. An injection device (100) according to claim 3, wherein the dosing label (9) can be plastically deformed in longitudinal direction, i.e., it maintains a C-form when folded.

7. An injection device (100) according to claim 1, wherein the dosing label (9) can be closed as a ring at the ends (911).

8. An injection device (100) according to claim 1, wherein the dosing label (9) includes one front face (92) and a back side (93) at least partially adhesive.

9. An injection device (100) according to claim 8, wherein the back side (93) is adhesive only at the ends (911).

10. An injection device (100) according to claim 8, wherein the dosing label (9) includes one central portion (910) placed between the two ends (911), and the length of the central portion (910) is smaller than the circumference of the plunger (5).

11. An injection device (100) according to claim 1, wherein the stem (53) of the plunger (5) has circular section.

12. An injection device (100) according to claim 1, wherein the base (52) of the plunger (5) projects laterally from the stem (53).

13. Production method of an injection device according to claim 1, including the steps of:
pre-arranging the tubular containment body (3), provided with containment chamber (33), and the plunger (5) slidingly engaged inside the tubular body (3);
pre-arranging the flexible dosing label (9);
applying the flexible dosing label (9) on the plunger (5) by means of a labeller.

14. An injection kit for injecting a controlled dose of an injectable solution, including:
an injection device (100), provided with a containment tubular body (3), that extends between one first end (31) and one second end (32); at least one containment chamber (33), defined inside the tubular body (3), for the containment of the injectable substance; a plunger (5) slidingly engaged inside the tubular body (3), provided with a central stem (53) placed between a cap (51) and a base (52), said cap (51) being slidingly arranged inside the containment chamber (33) to allow the outflow of the injectable substance;
at least one spacer applicable to the plunger (5) and adapted to allow a forward movement of the plunger (5) in the containment chamber (33) up to reach an abutment of the spacer between the base (52) of the plunger (5) and the second end (32) of the containment chamber (3) to avoid the further forward movement of the plunger (5) in the containment chamber (33);
characterized in that the spacer is a flexible dosing label (9), that can be deformed in one longitudinal direction (X) and rigid in one transverse direction (Y).

15. An injection kit according to claim 14, wherein the plunger (5) is graduated, i.e., it is provided with notches (55) at the stem (53) adapted to indicate to the user the correct positioning wherein the dosing label (9) must be applied.
